(19) United States
(12) Patent Application Publication
(10) Pub. No.: US 2016/0303113 A1
(43) Pub. Date: Oct. 20, 2016

(54) METHODS OF SELF-ADMINISTRATION OF VANOXERINE FOR TERMINATING ACUTE EPISODES OF CARDIAC ARRHYTHMIA IN MAMMALS

(71) Applicant: LAGUNA PHARMACEUTICALS ,INC., La Jolla, CA (US)

(72) Inventors: Arthur M. BROWN, Brecksville, OH (US); Howard C. DITTRICH, La Jolla, CA (US)

(21) Appl. No.: 14/781,288

(22) PCT Filed: Apr. 25, 2014
(86) PCT No.: PCT/US2014/035523

§ 371 Date: Sep. 29, 2015

Related U.S. Application Data
(60) Provisional application No. 61/816,214, filed on Apr. 26, 2013.
(60) Provisional application No. 61/904,796, filed on Nov. 15, 2013.
(60) Provisional application No. 61/905,017, filed on Nov. 15, 2013.
(60) Provisional application No. 61/915,792, filed on Dec. 13, 2013.
(60) Provisional application No. 61/915,829, filed on Dec. 13, 2013.

(51) Int. Cl. A61K 31/495

(52) U.S. Cl. CPC A61K 31/495

(57) ABSTRACT

Disclosed embodiments are related to methods of administration of vanoxerine wherein a patient, previously and successfully treated for cardiac arrhythmia with vanoxerine is prescribed a further dose for self-administration of a further dose of vanoxerine for treatment of a subsequent occurrence of cardiac arrhythmia.

### FIELD OF THE INVENTION

Presently disclosed embodiments are related to methods of treatment comprising administration of vanoxerine for terminating acute episodes of cardiac arrhythmia. Presently disclosed embodiments particularly relate to methods for dosing and treatment methodologies for self-administration of vanoxerine in the case of a re-occurrence of cardiac arrhythmia.

### BACKGROUND

Vanoxerine (1-[2-[bis(4-fluorophenyl)methoxy]ethyl]-4-(3-phenylpropyl)piperazine), its manufacture and/or certain pharmaceutical uses thereof are described in U.S. Pat. No. 4,202,896, U.S. Pat. No. 4,476,129, U.S. Pat. No. 4,874,765, U.S. Pat. No. 6,743,797 and U.S. Pat. No. 7,700,600, as well as European Patent EP 243,903 and PCT International Application WO 91/01732, each of which is incorporated herein by reference in its entirety.

Vanoxerine has been used for treating cocaine addiction, acute effects of cocaine, and cocaine cravings in mammals, as well as dopamine agonists for the treatment of Parkinsonism, acromegaly, hyperprolactinemia and diseases arising from a hypofunction of the dopaminergic system. (See U.S. Pat. No. 4,202,896 and WO 91/01732). Vanoxerine has also been used for treating and preventing cardiac arrhythmia in mammals. (See U.S. Pat. No. 6,743,797 and U.S. Pat. No. 7,700,600).

It is desirable to optimize methods of treatment using vanoxerine that provide for pre-determined dosing for self-administration of vanoxerine to treat subsequent occurrences of cardiac arrhythmia.

Atrial flutter and/or atrial fibrillation (AF) are the most commonly sustained cardiac arrhythmias in clinical practice, and are likely to increase in prevalence with the aging of the population. Currently, AF affects more than 1 million Americans annually, represents over 5% of all admissions for cardiovascular diseases and causes more than 80,000 strokes each year in the United States. In the US alone, AF currently afflicts more than 2.3 million people. By 2050, it is expected that there will be more than 12 million individuals afflicted with AF. While AF is rarely a lethal arrhythmia, it is responsible for substantial morbidity and can lead to complications such as the development of congestive heart failure or thromboembolism. Currently available Class I and Class III anti-arrhythmic drugs reduce the rate of re-occurrence of AF, but are of limited use because of a variety of potentially adverse effects, including ventricular proarrhythmia. Because current therapy is inadequate and fraught with side effects, there is a clear need to develop new therapeutic approaches.

Current first line pharmacological therapy options for AF include drugs for rate control. Despite results from several studies suggesting that rate control is equivalent to rhythm control, many clinicians believe that patients are likely to have better functional status when in sinus rhythm. Further, being in AF may introduce long-term mortality risk, where achievement of rhythm control may improve mortality.

Ventricular fibrillation (VF) is the most common cause associated with acute myocardial infarction, ischemic coronary artery disease and congestive heart failure. As with AF, current therapy is inadequate and there is a need to develop new therapeutic approaches.

Although various anti-arrhythmic agents are now available on the market, those having both satisfactory efficacy and a high margin of safety have not been obtained. For example, anti-arrhythmic agents of Class I, according to the classification scheme of Vaughan-Williams (“Classification of antiarrhythmic drugs,” Cardiac Arrhythmias, edited by: E. Sandoe, E. Flensted-Jensen, K. Olesen; Sweden, Astra, Sodertalje, pp 449-472 (1981)), which cause a selective inhibition of the maximum velocity of the upstroke of the action potential (Vₘₐₓ) are inadequate for preventing ventricular fibrillation because they shorten the wave length of the cardiac action potential, thereby favoring re-entry. In addition, these agents have problems regarding safety, i.e. they cause a depression of myocardial contractility and have a tendency to induce arrhythmias due to an inhibition of impulse conduction. The CAST (coronary artery suppression trial) study was terminated while in progress because the Class I antagonists had a higher mortality than placebo controls. β-adrenergenic receptor blockers and calcium channel (I_{Ca}) antagonists, which belong to Class II and Class IV, respectively, have a defect in that their effects are either limited to a certain type of arrhythmia or are contraindicated because of their cardiac depressant properties in certain patients with cardiovascular disease. Their safety, however, is higher than that of the anti-arrhythmic agents of Class I.

Prior studies have been performed using single dose administration of flecainide or propafenone (Class I drugs) in terminating atrial fibrillation. Particular studies investigated the ability to provide patients with a known dose of one of the two drugs so as to self-medicate should cardiac arrhythmia occur. P. Alboni, et al., “Outpatient Treatment of Recent-Onset Atrial Fibrillation with the ‘Pill-in-the-Pocket’ Approach,” NEJM 351; 23 (2004); L. Zhou, et al., “‘A Pill in the Pocket’ Approach for Recent Onset Atrial Fibrillation in a Selected Patient Group,” Proceedings of UCLA Healthcare 15 (2011). However, the use of flecainide and propafenone has been criticized as including candidates having structural heart disease and thus providing patients likely to have risk factors for stroke who should have received antithrombotic therapy, instead of the flecainide or propafenone. NEJM 352:11 (Letters to the Editor) (Mar. 17, 2005). Similarly, the use of warfarin concomitantly with propafenone was criticized.

Anti-arrhythmic agents of Class III are drugs that cause a selective prolongation of the action potential duration (APD) without a significant depression of the maximum upstroke velocity (Vₘₐₓ). They therefore lengthen the save length of the cardiac action potential increasing refractories, thereby antagonizing re-entry. Available drugs in this class are limited in number. Examples such as sotalol and amiodarone have been shown to possess interesting Class III properties (Singh B. N., Vaughan Williams E. M., “A Third Class of Anti-Arrhythmic Action: Effects on Atrial and Ventricular Intracellular Potentials and other Pharmacological Actions on Cardiac Muscle of MJ 1999 and AH 3747,” (Br. J. Pharmacol 39:675-689 (1970), and Singh B. N., Vaughan Williams E. M., “The Effect of Amiodarone, a New Anti-Anginal Drug, on Cardiac Muscle,” Br. J. Pharmacol 39:657-667 (1970)), but these are not selective Class III agents. Sotalol also possesses Class II (β-adrenergic blocking) effects which may cause cardiac depression and is contraindicated in certain susceptible patients.

Amiodarone also is not a selective Class III antiarrhythmic agent because it possesses multiple electrophysiological actions and is severely limited by side effects. (Nademanee, K., “The Amiodarone Odyssey,” J. Am. Coll. Cardiol. 20:1063-1065 (1992)). Drugs of this class are expected to be effective in preventing ventricular fibrillation. Selective Class III agents, by definition, are not considered to cause myocardial depression or an induction of arrhythmias due to inhibition of conduction of the action potential as seen with Class I antiarrhythmic agents.

Class III agents increase myocardial refractoriness via a prolongation of cardiac action potential duration (APD). Theoretically, prolongation of the cardiac action potential can be achieved by enhancing inward currents (i.e. Na+ or Ca²+ currents; hereinafter I_{Na} and I_{Ca}, respectively) or by reducing outward repolarizing potassium K+ currents. The delayed rectifier (I_{K}) K+ current is the main outward current involved in the overall repolarization process during the action potential plateau, whereas the transient outward (Iₜₒ) and inward rectifier (I_{KI}) K+ currents are responsible for the rapid initial and terminal phases of repolarization, respectively.

Cellular electrophysiologic studies have demonstrated that I_{K} consists of two pharmacologically and kinetically distinct K+ current subtypes, I_{Kr} (rapidly activating and deactivating) and I_{Ks} (slowly activating and deactivating). (Sanguinetti and Jurkiewicz, “Two Components of Cardiac Delayed Rectifier K+ Current. Differential Sensitivity to Block by Class III Anti-Arrhythmic Agents,” J Gen Physiol 96:195-215 (1990)). I_{Kr} is also the product of the human ether-a-go-go gene (hERG). Expression of hERG cDNA in cell lines leads to production of the hERG current which is almost identical to I_{Kr} (Curran et al., “A Molecular Basis for Cardiac Arrhythmia: hERG Mutations Cause Long QT Syndrome,” Cell 80(5):795-803 (1995)).

Class III anti-arrhythmic agents currently in development, including d-sotalol, dofetilide (UK-68,798), almokalant (H234/09), E-4031 and methanesulfonamide-N-[1′-6-cyano-1,2,3,4-tetrahydro-2-naphthalenyl)-3,4-dihydro-4-hydroxyspiro[2H-1-benzopyran-2, 4′-piperidin]-6y1], (+)-, monochloride (MK-499) predominantly, if not exclusively, block I_{Kr}. Although amiodarone is a blocker of I_{Ks} (Balser J. R. Bennett, P. B., Hondeghem, L. M. and Roden, D. M. “Suppression of time-dependent outward current in guinea pig ventricular myocytes: Actions of quinidine and amiodarone,” Circ. Res. 69:519-529 (1991)), it also blocks I_{Na} and I_{Ca}, effects thyroid function, as a nonspecific adrenergic blocker, acts as an inhibitor of the enzyme phospholipase, and causes pulmonary fibrosis (Nademanee, K., “The Amiodarone Odessey.” J. Am. Coll. Cardiol. 20:1063-1065 (1992)).

Reentrant excitation (reentry) has been shown to be a prominent mechanism underlying supraventricular arrhythmias in man. Reentrant excitation requires a critical balance between slow conduction velocity and sufficiently brief refractory periods to allow for the initiation and maintenance of multiple reentry circuits to coexist simultaneously and sustain AF. Increasing myocardial refractoriness, by prolonging APD, prevents and/or terminates reentrant arrhythmias. Most selective Class III antiarrhythmic agents currently in development, such as d-sotalol and dofetilide predominantly, if not exclusively, block I_{Kr}, the rapidly activating component of I_{K} found both in atria and ventricle in man.

Since these I_{Kr} blockers increase APD and refractoriness both in atria and ventricle without affecting conduction per se, theoretically they represent potential useful agents for the treatment of arrhythmias like AF and VF. These agents have a liability in that they have an enhanced risk of proarrhythmia at slow heart rates. For example, torsade de pointes, a specific type of polymorphic ventricular tachycardia which is commonly associated with excessive prolongation of the electrocardiographic QT interval, hence termed “acquired long QT syndrome,” has been observed when these compounds are utilized (Roden, D. M., “Current Status of Class III Antiarrhythmic Drug Therapy,” Am J. Cardiol, 72:44B-49B (1993)). The exaggerated effect at slow heart rates has been termed “reverse frequency-dependence” and is in contrast to frequency-independent or frequency-dependent actions. (Hondeghem, L. M., “Development of Class III Antiarrhythmic Agents,” J. Cardiovasc. Cardiol. 20 (Suppl. 2):S17-S22). The pro-arrhythmic tendency led to suspension of the SWORD trial when d-sotalol had a higher mortality than placebo controls.

The slowly activating component of the delayed rectifier (I_{Ks}) potentially overcomes some of the limitations of I_{Kr} blockers associated with ventricular arrhythmias. Because of its slow activation kinetics, however, the role of I_{Ks} in atrial repolarization may be limited due to the relatively short APD of the atrium. Consequently, although I_{Ks} blockers may provide distinct advantage in the case of ventricular arrhythmias, their ability to affect supra-ventricular tachyarrhythmias (SVT) is considered to be minimal.

Another major defect or limitation of most currently available Class III anti-arrhythmic agents is that their effect increases or becomes more manifest at or during bradycardia or slow heart rates, and this contributes to their potential for proarrhythmia. On the other hand, during tachycardia or the conditions for which these agents or drugs are intended and most needed, they lose most of their effect. This loss or diminishment of effect at fast heart rates has been termed “reverse use-dependence” (Hondeghem and Snyders, “Class III antiarrhythmic agents have a lot of potential but a long way to go: Reduced Effectiveness and Dangers of Reverse use Dependence,” Circulation, 81:686-690 (1990); Sadanaga et al., “Clinical Evaluation of the Use-Dependent QRS Prolongation and the Reverse Use-Dependent QT Prolongation of Class III Anti-Arrhythmic Agents and Their Value in Predicting Efficacy,” Amer. Heart Journal 126:114-121 (1993)), or “reverse rate-dependence” (Bretano, “Rate dependence of class III actions in the heart,” Fundam. Clin. Pharmacol. 7:51-59 (1993); Jurkiewicz and Sanguinetti, “Rate-Dependent Prolongation of Cardiac Action Potentials by a Methanesulfonanilide Class III Anti-Arrhythmic Agent: Specific Block of Rapidly Activating Delayed Rectifier K+ current by Dofetilide,” Circ. Res. 72:75-83 (1993)). Thus, an agent that has a use-dependent or rate-dependent profile, opposite that possessed by most current class III anti-arrhythmic agents, should provide not only improved safety but also enhanced efficacy.

Vanoxerine has been indicated for treatment of cardiac arrhythmias. Indeed, certain studies have looked at the safety profile of vanoxerine and stated that no side-effects should be expected with a daily repetitive dose of 50 mg of vanoxerine. (U. Sogaard, et. al., “A Tolerance Study of Single and Multiple Dosing of the Selective Dopamine Uptake Inhibitor GBR 12909 in Healthy Subjects,” International Clinical Psychopharmacology, 5:237-251 (1990)). However, Sogaard, et. al. also found that upon administration of higher doses of vanoxerine, some effects were seen with regard to concentration difficulties, increase systolic blood pressure, asthenia, and a feeling of drug influence, among other effects. Sogaard, et. al. also recognized that there were unexpected fluctuations in serum concentrations with regard to these healthy patients. While they did not determine the reasoning, control of such fluctuations may be important to treatment of patients.

Further studies have looked at the ability of food to lower the first-pass metabolism of lipophilic basic drugs, such as vanoxerine. (S. H. Ingwersen, et. al., “Food Intake Increases the Relative Oral Bioavailability of Vanoxerine,” Br. J. Clin. Pharmac; 35:308-130 (1993)). However, no methods have been utilized or identified for treatment of cardiac arrhythmias in conjunction with the modulating effects of food intake.

In view of the questions regarding safety and efficacy of current therapies, and certainly with regard to concurrent use of warfarin, a drug frequently administered as an anti-coagulant, to patients with risk for blood clots, among other disease, new methods are needed for administration of anti-arrhythmic medications. Accordingly, there is a need for improved methods using the pill-in-the-pocket approach using vanoxerine, for safe and efficacious, pre-determined doses for treatment of subsequent acute episodes of cardiac arrhythmia.

### SUMMARY

Embodiments of the present disclosure relate to methods for treating cardiac arrhythmias comprising: administering a first dose of vanoxerine to a mammal; measuring the physiological concentration of vanoxerine in said mammal; modifying the dosage of vanoxerine based on the measurement of the physiological concentration; and providing the modified dose of vanoxerine for administration to the same patient for self-administration upon a re-occurrence of cardiac arrhythmia.

Further embodiments of the present disclosure relate to methods for treating cardiac arrhythmias comprising: administering a first dose of vanoxerine to a patient; measuring the physiological concentration of vanoxerine in the patient; observing the effects of the dose on the patient; comparing the plasma level concentration to a pre-determined physiological concentration; modifying a second dose of vanoxerine; and providing the modified dose of vanoxerine to the patient for treatment of a subsequent episode of cardiac arrhythmia.

Other aspects of the present disclosure include methods for providing a pre-determined dose of vanoxerine for treatment of a re-occurrence of cardiac arrhythmia comprising; providing a pre-determined dose of vanoxerine to a patient who was previously, successfully administered vanoxerine for treatment of cardiac arrhythmia; and, instructing said patient to self-administer the pre-determined dose upon a subsequent episode of cardiac arrhythmia.

Other aspects of the present disclosure comprises methods for terminating acute episodes of cardiac arrhythmia, such as atrial fibrillation or ventricular fibrillation in a mammal, such as a human, by administering to that mammal at least an effective amount of vanoxerine to terminate an acute episode of cardiac arrhythmia; measuring the vanoxerine plasma levels in the subject; adjusting the dosage of vanoxerine based on pre-determined vanoxerine plasma levels; and providing the patient with an adjusted dose of vanoxerine for self-administration upon a subsequent episode of cardiac arrhythmia.

Other aspects of the present disclosure are directed to a method for restoring normal sinus rhythm in a mammal, such as a human, exhibiting cardiac arrhythmia by administering to that mammal at least an effective amount of vanoxerine to terminate an acute episode of cardiac arrhythmia; measuring the vanoxerine physiological concentration in the subject; adjusting the dosage of vanoxerine to meet a pre-determined vanoxerine physiological concentration; and providing the patient with an adjusted dose of vanoxerine for self-administration upon a subsequent episode of cardiac arrhythmia.

Other aspects of the present disclosure are directed to methods for preventing a re-occurrence of an episode of cardiac arrhythmia in a mammal, such as a human, by administering to that mammal at least an effective amount of vanoxerine to terminate an acute episode of cardiac arrhythmia; measuring the vanoxerine physiological level in the subject; determining an appropriate dose of vanoxerine based on pre-determined vanoxerine physiological levels; and providing the patient with an adjusted dose of vanoxerine for self-administration upon a subsequent episode of cardiac arrhythmia.

Other aspects of the present disclosure are directed to methods of providing a patient with a pre-determined dose of vanoxerine for use upon a future event of cardiac arrhythmia comprising: administering a first dose of vanoxerine to the patient to treat a first episode of cardiac arrhythmia; measuring the plasma level concentration of vanoxerine in the patient; comparing the measure dose of vanoxerine to a pre-determined target plasma level; determining an effective second dose of vanoxerine; and instructing the patient to self-administer the second dose of vanoxerine upon the occurrence of an episode of cardiac arrhythmia.

Other aspects of the present disclosure are directed to methods of treating a patient comprising: identifying a patient experiencing an episode of cardiac arrhythmia; administering vanoxerine to said patient thereby treating the episode of cardiac arrhythmia; prescribing a second course of vanoxerine to treat a second, subsequent episode of cardiac arrhythmia; and instructing the patient to self-administer the second course of vanoxerine upon the occurrence of a second episode of cardiac arrhythmia.

A further embodiment of the present disclosure relates to methods for preventing a re-occurrence of an episode of cardiac arrhythmia in a mammal, such as a human, by administering to that mammal at least an effective amount of a first composition comprising an effective amount of a first drug for treatment of cardiac arrhythmia, and administering to said same a patient an effective amount of a second composition comprising vanoxerine to prevent the re-occurrence of cardiac arrhythmia in said mammal.

Further embodiments of the present disclosure relate to methods for treating cardiac arrhythmias comprising: administering a first dose of vanoxerine to a patient; measuring the physiological concentration of vanoxerine in the patient; observing the effects of the dose on the patient; comparing the measured physiological concentrations to a pre-determined physiological concentration; determining an effective second dose of vanoxerine based on the difference between the pre-determined dose and the measured concentration; and prescribing the second dose of vanoxerine for subsequent administration to be held at or otherwise made available from a pharmacy until needed.

A further embodiment is a method for providing vanoxerine to a patient for treatment of an acute episode of cardiac arrhythmia comprising: ascertaining bioavailability of a first dose of vanoxerine received by the patient; prescribing or making available a second dose of vanoxerine in an amount targeted to achieve a desired physiological concentration of vanoxerine in the patient based on said bioavailability, to be provided to the patient upon occurrence of a subsequent event of cardiac arrhythmia, upon providing said second dose of vanoxerine to the patient, providing notification to a medical professional involved in the patient's care; and, optionally thereafter, prescribing or making available a third dose of vanoxerine to be provided to the patient upon a further event of cardiac arrhythmia.

A further embodiment is a method for treating a patient with vanoxerine to modulate plasma level concentrations of vanoxerine in a patient being treated for cardiac arrhythmia comprising: ascertaining vanoxerine bioavailability information based on a first dose of vanoxerine received by the patient; determining an effective second dose targeted to provide a desired therapeutic level of vanoxerine to the patient based on said bioavailability information; prescribing, or making available said modified second dose of vanoxerine to be self-adminstered by the patient upon occurrence of a subsequent episode of cardiac arrhythmia; instructing the patient to self-administer the second dose of vanoxerine upon the occurrence of an episode of cardiac arrhythmia.

A further embodiment is a method of prescribing vanoxerine to a patient for treatment of cardiac arrhythmia comprising: identifying a patient experiencing an episode of cardiac arrhythmia; administering vanoxerine to said patient thereby treating the episode of cardiac arrhythmia; prescribing a second course of vanoxerine to treat a second, subsequent episode of cardiac arrhythmia, wherein said second course of vanoxerine is provided to the patient; and instructing the patient to self-administer the second course of vanoxerine upon the occurrence of a second episode of cardiac arrhythmia.

A method of treating a patient comprising: identifying a patient experiencing an episode of cardiac arrhythmia; administering vanoxerine to said patient thereby treating the episode of cardiac arrhythmia; prescribing a second course of vanoxerine to treat a second, subsequent episode of cardiac arrhythmia; and instructing the patient to self-administer the second course of vanoxerine upon the occurrence of a second episode of cardiac arrhythmia.

A method of providing a patient with a pre-determined dose of vanoxerine for use upon a future event of cardiac arrhythmia comprising: administering a first dose of vanoxerine to the patient to treat a first episode of cardiac arrhythmia; measuring the physiological concentration of vanoxerine in the patient; modifying a second dose of vanoxerine; and instructing the patient to self-administer the second dose of vanoxerine upon the occurrence of an episode of cardiac arrhythmia.

A method for modulating plasma level concentrations in a patient being treated for cardiac arrhythmia comprising: administering a first dose of vanoxerine; measuring the plasma level concentration of vanoxerine in the patient; comparing the measured dose of vanoxerine to a pre-determined target plasma level; providing the patient with a further effective dose of vanoxerine; and instructing the patient to administer the further dose of vanoxerine upon re-occurrence of cardiac arrhythmia.

A method for administering vanoxerine to a patient for treatment of cardiac arrhythmia comprising: administering a first dose of vanoxerine; measuring the physiological concentration of vanoxerine; determining an effective second dose of vanoxerine based on the measured physiological concentration; providing a modified second dose to the patient; and instructing the patient to self-administer the second dose of vanoxerine upon the occurrence of an episode of cardiac arrhythmia.

Administering steps in any of the foregoing methods may comprise administration by a caregiver, a medical professional, or self-administered by a patient.

### DETAILED DESCRIPTION OF PREFERRED EMBODIMENTS

All references cited herein are hereby incorporated by reference in their entirety.

As used herein, the term “about” is intended to encompass a range of values ±10% of the specified value(s). For example, the phrase “about 20” is intended to encompass ±10% of 20, i.e. from 18 to 22, inclusive.

As used herein, the term “vanoxerine” refers to vanoxerine and pharmaceutically acceptable salts thereof.

As used herein, the term “pharmaceutically acceptable” refers to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable benefit/risk ratio.

As used herein, the term “subject” refers to a warm blooded animal such as a mammal, preferably a human or a human child, which is afflicted with, or has the potential to be afflicted with one or more diseases and conditions described herein.

As used herein, “therapeutically effective amount” refers to an amount which is effective in reducing, eliminating, treating, preventing or controlling the symptoms of the herein-described diseases and conditions. The term “controlling” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment.

As used herein, “unit dose” means a single dose which is capable of being administered to a subject, and which can be readily handled and packaged, remaining as a physically and chemically stable unit dose comprising either vanoxerine or a pharmaceutically acceptable composition comprising vanoxerine.

As used herein, “CYP3A4” means the cytochrome P450 3A4 protein, which is a monooxygenase that is known for its involvement in drug metabolism.

As used herein, “administering” or “administer” refers to the actions of a medical professional or caregiver, or alternatively self-administration by the patient.

The term “steady state” means wherein the overall intake of a drug is fairly in dynamic equilibrium with its elimination.

As used herein, a “pre-determined” plasma level or other physiological tissue or fluid and refers to a concentration of vanoxerine at a given time point. Typically, a pre-determined level will be compared to a measured level, and the time point for the measured level will be the same as the time point for the pre-determined level. In considering a pre-determined level with regard to steady state concentrations, or those taken over a period of hours, the pre-determined level is referring to the mean concentration taken from the area under the curve (AUC), as the drug increases and decreases in concentration in the body with regard to the addition of a drug pursuant to intake and the elimination of the drug via bodily mechanisms.

Cardiac arrhythmias include atrial, junctional, and ventricular arrhythmias, heart blocks, sudden arrhythmic death syndrome, and include bradycardias, tachycardias, re-entrant, and fibrillations. These conditions, including the following specific conditions: atrial flutter, atrial fibrillation, multifocal atrial tachycardia, premature atrial contractions, wandering atrial pacemaker, supraventricular tachycardia, AV nodal reentrant tachycardia, junctional rhythm, junctional tachycardia, premature junctional contraction, premature ventricular contractions, ventricular bigeminy, accelerated idioventricular rhythm, monomorphic ventricular tachycardia, polymorphic ventricular tachycardria, and ventricular fibrillation, and combinations thereof are all capable of severe morbidity and death if left untreated. Methods and compositions described herein are suitable for the treatment of these and other cardiac arrhythmias.

Interestingly, studies have identified that human subjects have significant variability with regard to the metabolism of vanoxerine. Vanoxerine, is susceptible to metabolism by CYP3A4 among other known P450 cytochromes. Accordingly, the bioavailability of a given dose of vanoxerine is impacted by certain P450 cytochromes. In particular, studies have identified that human subjects have variability with regard to metabolism which is predicted to be based on CYP3A4 and other P450 cytochromes. Typically, patients fall within one of two groups, a fast metabolism or a slow metabolism, such that the patients can be grouped with other patients and will have similar metabolic profiles for a given dose of vanoxerine. Patients in the fast metabolism group respond differently to vanoxerine than patients in the slow metabolism group with regard to Cₘₐₓ, tₘₐₓ, and AUC plasma concentrations as well as the half-life. Accordingly, it is possible to define whether a given patient is a fast or a slow metabolizer and predict their pharmacokinetic response to vanoxerine. Accordingly, determination of the patient's status within the fast or slow metabolic group can be utilized for improving efficacy and treatment of a patient.

Additionally, patients fall within a gradient within the slow and fast metabolism groups. Accordingly, there exists, even within the groupings, a continuum that provides that some people are faster or slower metabolizers even within the groups. Additional factors also play into the variability with regard to patient populations. Accordingly, when providing efficacious treatment for termination of cardiac arrhythmias, in some embodiments, it is important to determine or recognize where the patient falls within the spectrum of vanoxerine bioavailability, and provide a dose of vanoxerine that will be efficacious for that patient while also maximizing the safety profile of the drug.

Vanoxerine also has a moderately low oral bioavailability as a result of incomplete absorption and substantial first pass metabolism, from CYP3A4 and other p450 proteins. Vanoxerine is primarily eliminated from the body in urine, bile, and feces. Indeed, a substantial amount of the drug is expelled, unabsorbed into the feces. Additionally, pharmacokinetic parameters from tests in dogs suggest that there is a slow Tₘₐₓ of about 3 hours, low systemic bioavailability (23%) and slow elimination from the plasma (T₁₁₂ of 22 hours). However, the long half-life of the drug may actually be utilized to minimize the continuous or regular dosing of the drug.

Studies have questioned whether sustained, and/or chronic use of vanoxerine is suitable for mammalian patients. Preliminary studies have suggested that daily use of a drug over seven, 10, and 14 days may lead to increased heart rate and systolic blood pressure when taking concentrations of 75, 100, 125, and 150 mg of vanoxerine a day. However, control and prevention of events of cardiac arrhythmia are important to these patients to prevent future re-occurrences and the deleterious effects and morbidity.

Indeed, control and prevention of events of cardiac arrhythmia are important to these patients to prevent future re-occurrences and the deleterious effects and morbidity. One issue is that cardiac arrhythmia is a progressive disease and patients who suffer from a first cardiac arrhythmia are pre-disposed to suffering from additional episodes of cardiac arrhythmia. Any cardiac arrhythmia involves risk with regard to mortality and morbidity, and so terminating the cardiac arrhythmia in a timely and safe manner is a critical need for these patients, but preventing further events may be even more paramount.

Additional concerns for patients who have suffered from cardiac arrhythmia is compounding heart disease, as well as angina pectoris as well as other heart pain, chest pain, and other complications. Typically, concomitant use of an atrial fibrillation drug with a number of other drugs is contraindicated because of any number of interactions between the two drugs. However, certain drugs may establish a beneficial co-administration with vanoxerine wherein the concomitant administration of vanoxerine and at least one additional drug for treatment of cardiac arrhythmia allows for maintenance of steady state status of vanoxerine while providing for more frequent administration of said at least one additional drug. The combination allows for regular administration of vanoxerine to maintain normal sinus rhythm, but without the need for daily maintenance therapy, while providing for a dose of a second drug to be taken more frequently than the vanoxerine, to aiding the maintenance of normal sinus rhythm, and preventing further episodes of cardiac arrhythmia.

Therefore, upon an occurrence of cardiac arrhythmia, patients often visit an emergency room or other medical provider for administration of certain drugs that treat the cardiac arrhythmia, or other treatments, including ablation. However, it is not always feasible to quickly reach a doctor for fast, safe, and effective treatment of cardiac arrhythmia. Furthermore, in view of the dangers of some concomitant administration with other drugs, it is advantageous to provide patients who have previously suffered from a cardiac arrhythmia, and have successfully treated that cardiac arrhythmia with vanoxerine, with a further, measured dose of vanoxerine to treat a subsequent event of cardiac arrhythmia. Accordingly, a patient may take this measured dose home with them, carry it with them on travels, and, if an occurrence of cardiac arrhythmia occurs, they have a “pill-in-the-pocket” that will have been previously tested for treating that patient's cardiac arrhythmia.

A pill-in-the-pocket approach is intended to be a mechanism for providing a patient with a pre-determined, effective dose of vanoxerine to treat an occurrence of arrhythmia. Typically, a patient receiving the pill-in-the-pocket would have been previously, successfully administered vanoxerine for treatment of cardiac arrhythmia. During a first administration of vanoxerine, doctors are able to monitor the patient, either by watching the patient and seeing the responses to the drug, or through blood tests, or other physiological monitoring to review the safety profile and efficacy of the drug for the patient. Using the first administration as a test case allows a medical professional to then prescribe a future dose for self-administration to the patient upon re-ocurrence of an event of cardiac arrhythmia. This provides the patient with the ability to treat their own symptoms, regardless of their location and proximity to a hospital, if necessary.

Suitable methods for treatment of cardiac arrhythmias include various dosing schedules which may be administered by any technique capable of introducing a pharmaceutically active agent to the desired site of action, including, but not limited to, buccal, sublingual, nasal, oral, topical, rectal and parenteral administration. Dosing may include single daily doses, multiple daily doses, single bolus doses, slow infusion injectables lasting more than one day, extended release doses, IV or continuous dosing through implants or controlled release mechanisms, and combinations thereof. These dosing regimens in accordance with the method allow for the administration of the vanoxerine in an appropriate amount to provide an efficacious level of the compound in the blood stream or in other target tissues. Delivery of the compound may also be through the use of controlled release formulations in subcutaneous implants or transdermal patches.

For oral administration, a suitable composition containing vanoxerine may be prepared in the form of tablets, dragees, capsules, syrups, and aqueous or oil suspensions. The inert ingredients used in the preparation of these compositions are known in the art. For example, tablets may be prepared by mixing the active compound with an inert diluent, such as lactose or calcium phosphate, in the presence of a disintegrating agent, such as potato starch or microcrystalline cellulose, and a lubricating agent, such as magnesium stearate or talc, and then tableting the mixture by known methods.

Tablets may also be formulated in a manner known in the art so as to give a sustained release of vanoxerine. Such tablets may, if desired, be provided with enteric coatings by known method, for example by the use of cellulose acetate phthalate. Suitable binding or granulating agents are e.g. gelatine, sodium carboxymethylcellulose, methylcellulose, polyvinylpyrrolidone or starch gum. Talc, colloidal silicic acid, stearin as well as calcium and magnesium stearate or the like can be used as anti-adhesive and gliding agents.

Tablets may also be prepared by wet granulation and subsequent compression. A mixture containing vanoxerine and at least one diluent, and optionally a part of the disintegrating agent, is granulated together with an aqueous, ethanolic or aqueous-ethanolic solution of the binding agents in an appropriate equipment, then the granulate is dried. Thereafter, other preservative, surface acting, dispersing, disintegrating, gliding and anti-adhesive additives can be mixed to the dried granulate and the mixture can be compressed to tablets or capsules.

Tablets may also be prepared by the direct compression of the mixture containing the active ingredient together with the needed additives. If desired, the tablets may be transformed to dragees by using protective, flavoring and dyeing agents such as sugar, cellulose derivatives (methyl- or ethylcellulose or sodium carboxymethylcellulose), polyvinylpyrrolidone, calcium phosphate, calcium carbonate, food dyes, aromatizing agents, iron oxide pigments and the like which are commonly used in the pharmaceutical industry.

For the preparation of capsules or caplets, vanoxerine and the desired additives may be filled into a capsule, such as a hard or soft gelatin capsule. The contents of a capsule and/or caplet may also be formulated using known methods to give sustained release of the active compound.

Liquid oral dosage forms of vanoxerine may be an elixir, suspension and/or syrup, where the compound is mixed with a non-toxic suspending agent. Liquid oral dosage forms may also comprise one or more sweetening agent, flavoring agent, preservative and/or mixture thereof.

For rectal administration, a suitable composition containing vanoxerine may be prepared in the form of a suppository. In addition to the active ingredient, the suppository may contain a suppository mass commonly used in pharmaceutical practice, such as Theobroma oil, glycerinated gelatin or a high molecular weight polyethylene glycol.

For parenteral administration, a suitable composition of vanoxerine may be prepared in the form of an injectable solution or suspension. For the preparation of injectable solutions or suspensions, the active ingredient can be dissolved in aqueous or non-aqueous isotonic sterile injection solutions or suspensions, such as glycol ethers, or optionally in the presence of solubilizing agents such as polyoxyethylene sorbitan monolaurate, monooleate or monostearate. These solutions or suspension may be prepared from sterile powders or granules having one or more carriers or diluents mentioned for use in the formulations for oral administration. Parenteral administration may be through intravenous, intradermal, intramuscular or subcutaneous injections.

In effectively treating cardiac arrhythmia, it is necessary in some circumstances to provide for a certain plasma level concentration of vanoxerine. Plasma level concentrations are modified by the methods described herein. Patients have variability with regarding to their first pass metabolism of vanoxerine and so modification of the dose can provide an effective dose for administration to a patient. Plasma level concentrations, taken at a time point of 1 hour post administration are about 5 to about 1000 ng/ml. In alternative embodiments, plasma level concentrations at 1 hour post administration are about 10 to about 1000 ng/ml, or about 20 to about 400 ng/ml, or about 20 to about 200 ng/ml, or about 25 to about 150 ng/ml or about 40 to about 100 ng/ml, and about 60 to about 100 ng/ml.

In other embodiments, it is advantageous to provide for a certain dose, or a maximum dose at a given time point after administration of the vanoxerine to safely and effectively treat the cardiac arrhythmia. Accordingly, modification of Cₘₐₓ and tₘₐₓ is appropriate to maintain consistent Cₘₐₓ plasma level concentrations for a particular patient. Cₘₐₓ taken at a time point of 1 hour post administration are about 5 to about 1000 ng/ml. In alternative embodiments, plasma level concentrations at 1 hour post administration are about 10 to about 400 ng/ml, or about 20 to about 200 ng/ml, or about 20 to about 150 ng/ml, or about 25 to about 125 ng/ml or about 40 to about 100 ng/ml, and about 60 to about 100 ng/ml. Conversely tₘₐₓ is appropriately reached at about 1 hour post administration. In other embodiments, tₘₐₓ is appropriately reached at about 30 minutes, or about 90 minutes, or about 120 minutes, or about 240 minutes post administration. These maximum values vary widely by patient and modification of the dose, of the dosing schedule, of diet, and of other concomitant medications may be utilized to reach a predetermined therapeutic level.

In effectively treating cardiac arrhythmia, it is necessary in some circumstances to provide for a certain plasma level concentration of vanoxerine for efficacy. Plasma level concentrations are modified by the methods described herein. However, as patients have variability with regarding to their first pass metabolism of vanoxerine, modification of a subsequent dose can help ensure an appropriate and effective dose for administration to an individual patient, should modification be necessary. Effective plasma level concentrations for the treatment of cardiac arrhythima, taken at a time point of 1 hour post administration are about 5 to about 1000 ng/ml. In alternative embodiments, plasma level concentrations at 1 hour post administration are about 10 to about 400 ng/ml, or about 20 to about 200 ng/ml, or about 20 to 150 ng/ml, or about 40 to about 125 ng/ml or about 40 to 100 ng/ml.

In some embodiments, a dosage of 1 mg to 1000 mg per unit dose is appropriate to reach effective plasma concentrations. Other embodiments may utilize a dosage of about 50 mg to about 800 mg, or about 25 to about 100 mg, or about 100 mg to about 600 mg, or about 200 to about 400 mg. Preferred doses are about 25, 50, 75, 100, 150, 200, 300, and 400 mg.

### EXAMPLES

The materials, methods, and examples presented herein are intended to be illustrative, and not to be construed as limiting the scope or content of the invention. Unless otherwise defined, all technical and scientific terms are intended to have their art-recognized meanings.

Example 1: 28 patients participated in a study of vanoxerine. 25 patients took a 300 mg dose of vanoxerine and 3 patients took a placebo. Each patient gave samples before administration of their dose, and then again at nine further time points, 30 minutes after administration, 1, 2, 3, 4, 6, 8, 12, and 24 hours post administration.

TABLE 1
Concentrations ng/ml

| Time | | | | | | | Total |
|---|---|---|---|---|---|---|---|
| (h) | Vanoxerine | M03 | M04 | M01 | M02 | M05 | Metabolites |
| −15 | 1.00* | 1.00 | 1.00 | 1.00 | 1.00 | 1.00 | 1.00 |
| .5 | 25.26 | 1.02 | 10.79 | 1.93 | 1.00 | 1.30 | 12.44 |
| 1 | 70.09 | 2.46 | 49.74 | 7.51 | 1.02 | 1.88 | 60.41 |
| 2 | 104.98 | 7.08 | 82.62 | 19.65 | 1.02 | 2.59 | 111.20 |
| 3 | 81.43 | 7.21 | 75.63 | 18.68 | 1.01 | 2.14 | 102.83 |
| 4 | 54.30 | 7.54 | 63.85 | 16.42 | 1.01 | 1.45 | 88.35 |
| 6 | 32.85 | 6.59 | 48.14 | 11.48 | 1.00 | 1.22 | 66.35 |
| 8 | 24.37 | 4.92 | 38.38 | 8.98 | 1.00 | 1.21 | 52.45 |
| 12 | 15.89 | 3.98 | 26.84 | 6.30 | 1.00 | 1.05 | 37.05 |
| 24 | 8.29 | 2.32 | 13.46 | 3.66 | 1.00 | 1.01 | 19.07 |

*A quantity of (1.00) represents an amount that was below the lower limit of quantitation, which is <1.139 ng/ml vanoxerine, and <1.1141 ng/ml 17-hydroxyl vanoxerine.

TABLE 2
Standard Deviations

| Time | | | | | | | Total |
|---|---|---|---|---|---|---|---|
| (h) | Vanoxerine | M03 | M04 | M01 | M02 | M05 | Metabolites |
| −15 | 0.00 | 0.00 | 0.00 | 0.00 | 0.00 | 0.00 | 0.00 |
| .5 | 43.77 | 0.12 | 15.58 | 3.20 | 0.00 | 0.80 | 19.28 |
| 1 | 61.82 | 2.51 | 49.96 | 7.08 | 0.10 | 1.13 | 59.70 |
| 2 | 100.18 | 4.70 | 51.64 | 15.31 | 0.07 | 2.56 | 70.07 |
| 3 | 80.40 | 5.40 | 49.04 | 13.63 | 0.07 | 2.31 | 64.45 |
| 4 | 55.01 | 5.32 | 39.75 | 11.31 | 0.04 | 1.16 | 52.50 |
| 6 | 35.74 | 5.10 | 31.30 | 7.90 | 0.00 | 0.87 | 41.84 |
| 8 | 30.37 | 4.05 | 25.29 | 6.74 | 0.00 | 0.94 | 33.41 |
| 12 | 24.03 | 3.15 | 17.62 | 4.70 | 0.00 | 0.27 | 23.17 |
| 24 | 10.34 | 2.11 | 8.91 | 2.76 | 0.00 | 0.03 | 12.31 |

Table 2 shows the standard deviations from the above 25 patients receiving vanoxerine. The three patients receiving a placebo are not included in the data and all data points indicated levels of vanoxerine below the lower limit of quantitation.

Tables 1 and 2, above, show tests of 25 patients with a 300 mg dose of vanoxerine. Blood was drawn from each of the test patients before the administration of the vanoxerine, and then at 9 additional time points, one half hour after administration, then 1, 2, 3, 4, 6, 8, 12, and 24 hours subsequent to administration.

The 25 patients fall into two categories: 15 fell into a category of having the majority of time point levels that were below the average mean (as identified in Table 1) “low concentration group average,” and the remaining 10 patients had the majority of time points above the average mean “high concentration group average.”

TABLE 3
Low concentration group average:

| Time | | | | | | | Total |
|---|---|---|---|---|---|---|---|
| (h) | Vanoxerine | M03 | M04 | M01 | M02 | M05 | Metabolites |
| −15 | 1.00 | 1.00 | 1.00 | 1.00 | 1.00 | 1.00 | 1.00 |
| .5 | 16.99 | 1.00 | 12.17 | 1.52 | 1.00 | 1.37 | 13.39 |
| 1 | 40.07 | 2.78 | 56.35 | 6.76 | 1.03 | 1.73 | 66.46 |
| 2 | 42.50 | 6.48 | 74.06 | 14.09 | 1.00 | 1.30 | 94.80 |
| 3 | 31.40 | 5.36 | 59.58 | 11.38 | 1.00 | 1.14 | 76.25 |
| 4 | 24.40 | 5.91 | 51.98 | 10.34 | 1.00 | 1.05 | 68.14 |
| 6 | 16.69 | 4.96 | 38.61 | 7.08 | 1.00 | 1.00 | 50.52 |
| 8 | 11.82 | 3.29 | 29.92 | 5.30 | 1.00 | 1.00 | 38.45 |
| 12 | 6.31 | 2.58 | 20.60 | 3.67 | 1.00 | 1.00 | 26.71 |
| 24 | 5.01 | 1.79 | 12.09 | 2.66 | 1.00 | 1.00 | 16.08 |

TABLE 4
Low concentration standard deviation:

| Time | | | | | | | Total |
|---|---|---|---|---|---|---|---|
| (h) | Vanoxerine | M03 | M04 | M01 | M02 | M05 | Metabolites |
| −15 | 0.00 | 0.00 | 0.00 | 0.00 | 0.00 | 0.00 | 0.00 |
| .5 | 0.00 | 0.00 | 0.00 | 0.00 | 0.00 | 0.00 | 0.00 |
| 1 | 24.47 | 0.00 | 17.68 | 1.67 | 0.00 | 0.98 | 20.45 |
| 2 | 27.50 | 3.10 | 59.32 | 7.56 | 0.13 | 1.05 | 71.04 |
| 3 | 28.16 | 4.18 | 44.96 | 9.05 | 0.00 | 0.58 | 57.77 |
| 4 | 22.66 | 3.28 | 34.95 | 7.06 | 0.00 | 0.46 | 45.53 |
| 6 | 16.11 | 3.72 | 30.77 | 7.28 | 0.00 | 0.16 | 42.04 |
| 8 | 14.20 | 3.51 | 21.42 | 3.71 | 0.00 | 0.00 | 28.30 |
| 12 | 11.19 | 2.27 | 15.60 | 2.86 | 0.00 | 0.00 | 20.34 |
| 24 | 3.07 | 1.69 | 10.44 | 1.72 | 0.00 | 0.00 | 13.40 |

TABLE 5
High concentration group average:

| Time | | | | | | | Total |
|---|---|---|---|---|---|---|---|
| (h) | Vanoxerine | M03 | M04 | M01 | M02 | M05 | Metabolites |
| −15 | 1.00 | 1.00 | 1.00 | 1.00 | 1.00 | 1.00 | 1.00 |
| .5 | 37.67 | 1.06 | 8.71 | 2.55 | 1.00 | 1.19 | 11.01 |
| 1 | 115.12 | 1.98 | 39.82 | 8.64 | 1.00 | 2.10 | 51.33 |
| 2 | 198.71 | 7.96 | 95.46 | 28.00 | 1.05 | 4.51 | 135.79 |
| 3 | 156.49 | 9.98 | 99.70 | 29.64 | 1.03 | 3.64 | 142.69 |
| 4 | 96.14 | 9.83 | 80.45 | 24.93 | 1.02 | 2.01 | 116.64 |
| 6 | 57.08 | 9.03 | 62.44 | 18.08 | 1.00 | 1.55 | 90.10 |
| 8 | 43.18 | 7.37 | 51.08 | 14.50 | 1.00 | 1.52 | 73.46 |
| 12 | 29.30 | 5.93 | 35.57 | 9.98 | 1.00 | 1.13 | 51.52 |
| 24 | 3.07 | 1.69 | 10.44 | 1.72 | 0.00 | 0.00 | 13.40 |

TABLE 6
High concentration group standard deviation:

| Time | | | | | | | Total |
|---|---|---|---|---|---|---|---|
| (h) | Vanoxerine | M03 | M04 | M01 | M02 | M05 | Metabolites |
| −15 | 0.00 | 0.00 | 0.00 | 0.00 | 0.00 | 0.00 | 0.00 |
| .5 | 62.39 | 0.19 | 12.37 | 4.71 | 0.00 | 0.45 | 18.34 |
| 1 | 72.52 | 1.19 | 31.62 | 6.52 | 0.00 | 1.26 | 38.76 |
| 2 | 96.23 | 5.50 | 60.49 | 19.21 | 0.11 | 3.17 | 82.34 |
| 3 | 77.51 | 6.85 | 58.66 | 13.99 | 0.11 | 3.12 | 70.07 |
| 4 | 63.43 | 6.50 | 46.33 | 10.60 | 0.06 | 1.67 | 54.47 |
| 6 | 44.79 | 6.26 | 38.98 | 8.02 | 0.00 | 1.35 | 48.76 |
| 8 | 40.12 | 4.97 | 32.08 | 7.21 | 0.00 | 1.48 | 38.93 |
| 12 | 33.45 | 3.74 | 22.14 | 5.13 | 0.00 | 0.42 | 26.71 |
| 24 | 14.82 | 3.02 | 11.03 | 3.24 | 0.00 | 0.05 | 14.70 |

As can be seen, in Tables 3 and 5, the low concentration group barely has plasma levels rise above 40 ng/ml at any time point in reference to vanoxerine. Whereas, the high concentration group has levels that rise to nearly 200 ng/ml at a time of two (2) hours after administration. Furthermore, the variability with regard to each of the groups is also wider. The standard deviations in Table 4 are lower than those in Table 6, (no T-test or 95% confidence was run), demonstrating that the variability was greater in the high concentration group than the low concentration group.

Example 2: 12 subjects received daily doses of Vanoxerine for 11 consecutive days, at doses of 25, 50, 75, and 100 mg, with a 14 day washout period between dose levels.

At 25 mg, plasma levels were not detectable after 8 hours. At 50, 75, and 100 mg doses, plasma levels were detectable at 24 hours and steady state was reached by day 8. PK was linear and dose proportional across 50, 75 and 100 mg doses. The 100 mg QD Cₘₐₓₛₛ and AUC₀₋₂₄ₛₛ suggests a trend toward non-linear PK that may become apparent at doses >100 mg QD. PK was highly variable at steady-state; Cₘₐₓ, ss, and AUC₀₋₂₄ₛₛ inter-subject variability ranged from 55-85%. The results are listed below in Table 7.

TABLE 7

| | PK Data | |
|---|---|---|
| | (Mean +/− SD) | PK Data (Mean +/− SD) |
| Dose | C_{Max} | T_{1/2} |
| 50 mg | 27.5 +/ 21.3 ng/ml | 49.39 +/− 26.18 hr (4.71-110.57) |
| T_{Max} | | |
| 1.27 +− 0.5 hr | | |
| (0.5-2.0) | | |
| 75 mg | 27.4 +/− 15.5 ng/ml | 52.53 +/− 37.46 (10.26-116.67) |
| 100 mg | 40.2 +/− 26.6 ng/ml | 15.38 +/− 43.55 (5.56-125.00) |

Data from these studies demonstrates an increased half-life of the drug when daily doses are given. Furthermore, it was noted that heart rate and systolic blood pressure increased slightly in most subjects at 75 and 100 mg doses and did not completely return to baseline during washout between dose levels.

### Example 3

Fourteen healthy patients were given vanoxerine of 25, 75, and 125 mg, daily, for 14 days with a washout of 14 days between dose levels. A standardized meal was served 15 minutes prior to each dosing.

No significant adverse events were seen in any of the studies. Steady-state serum levels were reported within 9-11 days with disproportionately and statistically greater levels at higher doses as compared with the lower doses. The non-linear kinetics may be due to increasing bioavailability at higher doses based on a saturation of first pass metabolism. Indeed, a small proportion of the absorbed dose appears to undergo enterohepatic circulation, which may account for the considerably longer elimination half-life observed with repetitive dosing (22 hours versus 66 hours for single versus repeat administration). Steady-state plasma concentrations were achieved after 3 days with repetitive dosing, though all patients achieved steady state after 7-10 days of dosing, suggesting little potential for accumulation. This is supported by the observed lack of differential effects observed following single or repeat administration in pharmacology studies and in the absence of an effect on receptor number with repeat vanoxerine doses.

### Example 4

Four patients were given 50, 100, and 150 mg vanoxerine, daily, for 7 days.

Upon administration of 100 mg for 7 days, increases in systolic blood pressure and heart rate were seen. Similarly, during the 150 mg test, the patients also saw increases in systolic blood pressure and in heart rate. Steady-state levels were achieved within one week for all patients

### Example 5

In certain canine dosing models, adult mongrel dosages, 18-23 kg, were give oral vanoxerine. Three doses were given, 90 mg at 0 min, 180 mg at 60 min, and 270 mg at 120 min. Vanoxerine plasma concentration was measured against time, and it was tested at what concentration was there an inability to reinduce atrial flutter or atrial fibrillation. All dogs studied found the inability to reintroduce AF or AFL at concentrations between 70 and 105 ng/ml.

### Example 6

3 different cohorts, each including 35 subjects were enrolled in a study with 25 taking vanoxerine and 10 receiving placebo. Cohort 1 included 200 mg vanoxerine, Cohort 2 include 200 or 300 mg of vanoxerine, and Cohort 3 included 200, 300, or 400 mg vanoxerine. The vanoxerine or identical appearing placebo was randomly assigned and administered in a double-blinded fashion.

TABLE 8
Atrial Fibrillation/Flutter history:

| | Placebo (32) | 200 mg (22) | 300 mg (25) | 400 mg (25) |
|---|---|---|---|---|
| A Flutter at | 4 (12.5) | 4 (18.2) | 4 (16) | 4 (16 |
| Entry N (%) | | | | |

Duration of Concurrent AF/AFL Episode

| | | | | |
|---|---|---|---|---|
| Mean, days | 1.84 | 2.33 | 2.43 | 4 1.97 |
| range, days | 0-6 | 0-6 | 0-6 | 0-7 |
| Rx same day as | 41 | 23 | 32 | 32 |
| onset, % | | | | |

Time since AF/AFL Dx

| | | | | |
|---|---|---|---|---|
| Mean, yrs | 3.9 | 4.8 | 4.5 | 5.1 |
| range, yrs | 0-21 | 0-13 | 0-13 | 1-13 |
| Rx prior DC | 44 | 45 | 52 | 32 |
| cadioversion % | | | | |

Time since last DC Cardioversion

| | | | | |
|---|---|---|---|---|
| Mean, mo | 13.6 | 15.2 | 18.2 | 21 |
| range, mo | 0-77 | 0-5 | 0-90 | 0-103 |

TABLE 9
Efficacy: Percent conversion through 4, 8, and 24 hours

| | Placebo (32) | 200 mg (22) | 300 mg (25) | 400 mg (25) |
|---|---|---|---|---|
| 0-4 hr | 13% | 18% | 40% | 52% |
| 0-8 hr | 23% | 45% | 52% | 76% |
| 0-24 hr | 38% | 59% | 64% | 84% |

Indeed, there is a significant improvement in conversion as compared to placebo at all time-points, wherein the rate of conversion or percent conversion at 0-4 hours, 0-8 hours and 0-24 hours was improved with any dose of vanoxerine. Accordingly, a measurement of the improvement comprises a comparison to the rate of conversion of placebo, wherein the improvement is based on the percent increase in conversion over placebo. The 200 mg, having an improvement of conversion of 38%, 96%, and 55% at the above time points, 300 mg: 207%, 126%, and 68%, and the 400 mg: 300%, 230%, and 121%.

TABLE 10
Time to conversion

| Log-rank test results for time conversion | P-value |
|---|---|
| Overall | 0.0005 |
| Pairwise: 200 mg versus control | 0.0838 |
| pairwise: 300 mg versus control | 0.0180 |
| pairwise: 400 mg versus control | <0.0001 |

Indeed, the time to conversion based on the P-value and the above chart provides that placebo does not have greater than a 40% conversion at any time point below 24 hours, whereas all doses of vanoxerine are greater than 40% conversion at about 7 hours, and conversion greater than 50% for all dose at 12 hours, and nearing 60% at about 16 hours.

TABLE 11
Conversion of Atrial Flutter

| | Placebo (32) | 200 mg (22) | 300 mg (25) | 400 mg (25) |
|---|---|---|---|---|
| A flutter, N | 4 | 4 | 4 | 4 |
| Conversion, % | 25% | 50% | 75% | 75% |

Definition of “pure” atrial flutter: only Atrial Flutter (no AF) seen at −30, −15, and 0 time points. Conversion at any time within 24 hours. No 1:1 AFL seen post dose in any subject.

TABLE 12
Adverse events:

| Placebo (32) | 200 mg (22) | 300 mg (25) | 400 mg (25) |
|---|---|---|---|
| 7 (22%) subjects | 4 (18%) subjects | 7 (28%) subjects | 10 (40%) |
| reporting 10 | reporting 8 AEs | reporting 12 AEs | subjects |
| AE's | (1 SAE) | | reporting 23 |
| | | | AEs (1 SAE) |

In view of doses of 200, 300 and 400 mg, there was a highly statistically significant dose dependent increase in the conversion to sinus rhythm of recent onset symptomatic AF/AFL. The highest oral dose of 400 mg achieved a conversion rate of 76% at 8 hours and 84% within 24 hours. Time to conversion curves also demonstrate increasing slope of conversion with successively higher doses, suggesting a Cₘₐₓ dependent effect.

Vanoxerine was well tolerated at all doses with only two serious adverse events, one at the 200 mg dose and one at the 400 mg dose (the 200 mg dose being an upper respiratory infection, the 400 mg dose being lower extremity edema secondary to amlodipine), neither related to the study drug. Similar to efficacy, there was a dose dependent increase in adverse events, but only the high dose event rate was notably higher than that of the placebo group. Accordingly, vanoxerine has a high degree of efficacy for the conversion of recent onset symptomatic atrial fibrillation and atrial flutter in the absence of proarrhythmia, wherein the conversion rate approaches that of DC cardioversion.

Accordingly, hemodynamic effects on heart rate and systolic blood pressure have been seen with multiple dosing of vanoxerine. Several subjects exhibited dose-related increases in heart rate and systolic blood pressure. These effects, however, do not correlate with vanoxerine concentration AUC and interpretation is further confounded by the lack of placebo-control. These effects do not immediately dissipate upon discontinuation of study drug. It is suggested that vanoxerine exerts an effect on the autonomic nervous system over the course of the study. The lack of correlation with plasma vanoxerine AUC, may be interpreted as either evidence of a significant pharmacodynamic lag in the hemodynamic effects of vanoxerine or evidence that a metabolite is responsible for the hemodynamic effects.

This study favorable compares favorably to human models where plasma concentration is charted against conversion to normal sinus rhythm. Patients that failed to convert to normal sinus rhythm had concentration of vanoxerine was between 0 ng/ml and 40 ng/ml. Conversely, patients that conversed had vanoxerine concentrations between about 30 and 130 ng/ml, with a few outliers on the low end and high end. However, most conversions occurred in the range of about 60 ng/ml. Accordingly, modifying doses to reach 60 ng/ml or higher is suggested for effective conversion to normal sinus rhythm.

Accordingly, hemodynamic effects on heart rate and systolic blood pressure have been seen with multiple dosing of vanoxerine. Several subjects exhibited dose-related increases in heart rate and systolic blood pressure. These effects, however, do not correlate with vanoxerine concentration AUC and interpretation is further confounded by the lack of placebo-control. These effects do not immediately dissipate upon discontinuation of study drug. It is suggested that vanoxerine exerts an effect on the autonomic nervous system over the course of the study. The lack of correlation with plasma vanoxerine AUC, may be interpreted as either evidence of a significant pharmacodynamic lag in the hemodynamic effects of vanoxerine or evidence that a metabolite is responsible for the hemodynamic effects.

In view of the variability of patients, it may be advantageous to determine the profile of the patient such that subsequent administration of vanoxerine is determined by the pharmacokinetic profile of the individual patient, wherein a given dose will reach effective plasma concentrations. Accordingly, the method comprises administration of vanoxerine in a patient to meet a pre-determined therapeutic level at a given time-point; measurement of the plasma level post administration; providing a further administration to increase plasma level concentration, if the pre-determined therapeutic level is not reached. This allows for safe and effective treatment of the patient.

Accordingly, because of the known variability within the patient population and the need to optimize a treatment for patients suffering from a re-occurrence of cardiac arrhythmia, it is necessary to create methods for treating a patient suffering from cardiac arrhythmia with a prescribed dose of vanoxerine effective to treat the cardiac arrhythmia; subsequent to the administration of a first dose of vanoxerine, measuring the plasma levels of vanoxerine and/or one or more metabolites of vanoxerine; modifying a subsequent dose to provide a dose of vanoxerine closer to the pre-determined plasma levels than the first administered dose; and instructing the patient to self-administer the modified subsequent dose upon a subsequent episode of cardiac arrhythmia. In some embodiments, the modified dose is determined by comparing the measured plasma levels to a pre-determined plasma level concentrations of vanoxerine and/or the one or more metabolites

Modulation of a dose provides for greater accuracy with regard to target plasma concentrations for the treatment of cardiac arrhythmia. A first effective dose of vanoxerine provides for data to properly calibrate a future dose of vanoxerine, and allows for appropriate modulation of Cₘₐₓ and tₘₐₓ such that an effective plasma concentration of vanoxerine and/or one or more metabolite is reached for safe and effective treatment of the cardiac arrhythmia. Therefore, the methods provided for herein, provide for greater accuracy with regard to providing effective doses for reaching target plasma levels effective for treating cardiac arrhythmias and restoring patients to normal sinus rhythm, thus increasing the safety profile, improving efficacy of treatment, and minimizing side effects that may be associated with treatment. Accordingly, a physiological concentration at a time of between 1 and 4 hours post administration of between about 20 and 150 ng/ml, and more preferable between about 60 and 130 ng/ml is efficacious for most patients to convert to normal sinus rhythm.

Although the present invention has been described in considerable detail, those skilled in the art will appreciate that numerous changes and modifications may be made to the embodiments and preferred embodiments of the invention and that such changes and modifications may be made without departing from the spirit of the invention. It is therefore intended that the appended claims cover all equivalent variations as fall within the scope of the invention.

What is claimed is:

1. A method for administering vanoxerine to a patient for treatment of cardiac arrhythmia comprising: administering a first dose of vanoxerine; measuring the physiological concentration of vanoxerine; determining an effective second dose of vanoxerine based on the measured physiological concentration; providing an effective second dose to the patient; and instructing the patient to self-administer the second dose of vanoxerine upon the occurrence of an episode of cardiac arrhythmia.

2. The method of claim 1 further comprising the step of comparing the measure dose of vanoxerine to a pre-determined target physiological concentration.

3. The method of claim 2 wherein the pre-determined target physiological concentration is based on the plasma concentration.

4. The method of claim 3 wherein the target physiological concentration is between 20 and 150 ng/ml at a time between 1 and 4 hours post administration.

5. The method of claim 3 wherein the target physiological concentration is between 60 and 130 ng/ml at a time between 1 and 4 hours post administration.

6. A method for modulating plasma level concentrations in a patient being treated for cardiac arrhythmia comprising: administering a first dose of vanoxerine; measuring the plasma level concentration of vanoxerine in the patient; comparing the measured dose of vanoxerine to a pre-determined target plasma level; providing the patient with a further effective dose of vanoxerine; and instructing the patient to administer the further dose of vanoxerine upon re-occurrence of cardiac arrhythmia.

7. The method of claim 6 wherein the target plasma concentration is between 40 and 150 ng/ml.

8. The method of claim 6 wherein the target plasma level concentration is between 60 and 120 ng/ml.

9. A method of providing a patient with a pre-determined dose of vanoxerine for use upon a future event of cardiac arrhythmia comprising: administering a first dose of vanoxerine to the patient to treat a first episode of cardiac arrhythmia; measuring the physiological concentration of vanoxerine in the patient; modifying a second dose of vanoxerine; and instructing the patient to self-administer the second dose of vanoxerine upon the occurrence of an episode of cardiac arrhythmia.

10. The method of claim 10 wherein the physiological concentration is measured in the plasma.

11. The method of claim 10 further comprising the step of comparing the measured dose of vanoxerine to a pre-determined target plasma level.

12. The method of claim 11 wherein the target plasma level is between 20 and 150 ng/ml.

13. The method of claim 11 wherein the target plasma level is between 60 and 130 ng/ml.

14. A method of treating a patient comprising:
a. identifying a patient experiencing an episode of cardiac arrhythmia;
b. administering vanoxerine to said patient thereby treating the episode of cardiac arrhythmia;
c. prescribing a second course of vanoxerine to treat a second, subsequent episode of cardiac arrhythmia; and
d. instructing the patient to self-administer the second course of vanoxerine upon the occurrence of a second episode of cardiac arrhythmia.

15. The method of claim 14 further comprising the step of measuring the patients pharmacokinetic response to vanoxerine after the administration of the first dose of vanoxerine.

16. The method of claim 14, wherein the pharmacokinetic response is measured in the plasma of the patient.

17. The method of claim 16, further comprising the step of comparing the plasma level of the patient to a pre-determined effective plasma concentration.

18. The method of claim 17 wherein the pre-determined effective plasma concentration is between 20 and 150 ng/ml.

19. The method of claim 17 wherein the pre-determined effective plasma concentration is between 60 and 130 ng/ml.

20. The method of claim 17 wherein the pre-determined effective plasma concentration is at least 60 ng/ml.
